SEQUENCE LISTING (5 sequences)

SEQ ID NO: 1 - protein, 21 aa, Artificial Sequence
  Description of Artificial Sequence Synthetic peptide
KSLSLSLRGSLSLSLKGRGDS

SEQ ID NO: 2 - protein, 16 aa, Artificial Sequence
  Description of Artificial Sequence Synthetic peptide
KSLSLSLRGSLSLSLK

SEQ ID NO: 3 - protein, 4 aa, Artificial Sequence
  Description of Artificial Sequence Synthetic peptide
GGSG

SEQ ID NO: 4 - protein, 6 aa, Artificial Sequence
  Description of Artificial Sequence Synthetic peptide
GFPGER

SEQ ID NO: 5 - protein, 4 aa, Artificial Sequence
  Description of Artificial Sequence Synthetic peptide
RGDS

(19) United States
(12) Patent Application Publication
(10) Pub. No.: US 2017/0172953 A1
(43) Pub. Date: Jun. 22, 2017

(54) SELF-ASSEMBLING DRUG DELIVERY VEHICLES WITH IONICALLY CROSS-LINKED DRUGS

(71) Applicant: WILLIAM MARSH RICE UNIVERSITY, HOUSTON, TX (US)

(72) Inventors: Jeffrey Dale Hartgerink, Pearland, TX (US); Vivek Ashok Kumar, Houston, TX (US); Abhishek A. Jalan, Houston, TX (US); I-Che Li, Houston, TX (US)

(21) Appl. No.: 15/449,119

(22) Filed: Mar. 3, 2017

Related U.S. Application Data
(63) Continuation of application No. PCT/US15/48092, filed on Sep. 2, 2015.
(60) Provisional application No. 62/045,053, filed on Sep. 3, 2014.

(51) Int. Cl. A61K 31/185; C07K 14/00

(52) U.S. Cl. CPC A61K 31/185; A61K 47/48246; A61K 47/48784; C07K 14/001; C07K 2319/50

(57) ABSTRACT

A composition and system for delivering a therapeutic agent is provided. The composition includes a self-assembling peptide ionically cross-linked with a charged therapeutic agent. In the presence of the charged therapeutic agent, the peptide self-assembles to form a nanofibrous hydrogel scaffold, wherein the hydrogel structure is quickly recoverable following a shear stress thereby permitting administration of the composition by syringe-needle or catheter injection. Methods of using the composition is also provided.

### CROSS-REFERENCE TO RELATED APPLICATIONS

The present application is a continuation application of International Application No. PCT/US15/48092, filed Sep. 2, 2015, which claims priority to application claims the benefit of U.S. Provisional Application No. 62/045,053 filed on Sep. 3, 2014, the contents of which are incorporated herein by reference.

### STATEMENT OF GOVERNMENT INTEREST

This invention was made with government support under Grant Numbers R01 DE021798 and F32 DE023696 awarded by the National Institute of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 2, 2015, is named 14-21025-WO(260947.00257)_SL.txt and is 1,557 bytes in size.

### BACKGROUND

Treatment options for neoplastic disease typically involve radiation, chemotherapeutics and non-invasive/minimally-invasive treatments if diagnosed in early stages. For late-stage solid tumors (eg. glioblastoma multiforme), treatment involves surgical resection, coupled with post-operative radiation and/or chemotherapy. Systemic chemotherapeutics are typically used in high concentrations to ensure that malignancies in the far reaches of the extracellular space still receive the minimum effective therapeutic dosage. This comes at the cost of systemic side effects, resulting in significant patient discomfort, morbidity and mortality. To obviate this, localized delivery systems with therapeutic payloads can be administered peri-operatively in situ during post-surgical resection to reduce the immune compromising chemotherapeutic burden, permit targeted chemotherapy, and reduce localized malignancy.

One class of localized delivery system includes multi-domain peptides (MDP). MDPs are peptide amphiphiles that have the ability to self-assemble as a function of amino acid composition and ionic buffers (eg. Mg²⁺/Ca²⁺/PO₄³⁻). At neutral pH, protonated amine side groups of positively-charged residues align and resist molecular self-assembly of growing fibers, yielding a phenomenon known as molecular frustration. The addition of high ionic strength buffers or polyvalent anions can specifically aid in relieving molecular frustration, shielding positive or negative charges, allowing formation of long-range microscopic fibers. The present disclosure provides a therapeutic delivery system wherein the therapeutic agent can aid in relieving molecular frustration through ionic crosslinking with an MDP thereby providing a strong hydrogel with controlled, long-term release profile.

### SUMMARY

A composition and system for delivery of therapeutic agents is provided. In one embodiment, the composition comprises peptides ionically cross-linked with a charged therapeutic compound.

In certain embodiments, the peptides making up the composition comprise two or more domains. A first domain comprises one to four negatively or positively charged amino acids. In this embodiment, the first domain is located at both the N-terminus and C-terminus of a second domain. The second domain comprises two to six repeats of an amino acid sequence consisting of a hydrophilic amino acid and a hydrophobic amino acid (i.e., alternating hydrophobic and hydrophilic amino acids). The second domain drives the self-assembly of the peptide into a β-sheet structure through hydrophobic interactions and hydrogen bonds. In certain embodiments, each peptide may further comprise a spacer, a cell adhesion sequence, and/or an enzymatic cleavage signaling sequence. It certain embodiments, at least four peptides interact to form a peptide nanofiber. These peptides interact to form hydrogels.

The composition further comprises a charged therapeutic compound. The charged therapeutic compound ionically interacts with the charged amino acids (first domain) of the peptides to further promote long-range fiber growth, entanglement and hydrogel formation. Additionally, the ionic interaction between the charged therapeutic agent and the peptides further aid in sequestration and controlled release of the charged therapeutic agent from the hydrogel thereby providing an extended release composition.

A method is also provided. In one embodiment, the method comprises administering a therapeutic agent to a target tissue of a subject diagnosed with a disease, wherein the therapeutic agent is administered in an extended-release composition comprising the peptides described above and is selected to treat the disease. Due to the properties of the extended-release composition, the step of administering the therapeutic agent can be performed non-invasively via injection and due to the controlled release provided by the composition, may only need to be administered once every 2-4 weeks or longer.

### DRAWINGS

The patent or application file contains at least one drawing executed in color. Copies of this patent or patent application publication with color drawings will be provided by the office upon request and payment of the necessary fee.

A more complete understanding of this disclosure may be acquired by referring to the following description taken in combination with the accompanying figures.

FIG. 1 provides a schematic of molecular self-assembly of the one embodiment of the present composition comprising suramin as the cross-linking therapeutic agent. Here, the alternating hydrophobic/hydrophilic region (serine-leucine repeats) arranges antiparallel to each other via hydrophobic interactions and hydrogen bonds to form sheets. Suramin allows for intra and inter molecular hydrogen bonding and ionic interaction with terminal charged residues (e.g. lysine). Stabilized short fibers' terminal charges are screened allowing fiber growth and polymerization into hydrogel networks.

FIG. 2 provides the chemical structures of several exemplary charged therapeutic agents, namely suramin, clodronate, and tryphan blue, that may be used in certain embodiments of the present compositions.

FIG. 3 provides a graphical representation of rheological properties of cross-linked peptide hydrogels. Higher concentrations of negative ion charge shield MDP charges to a greater extent yielding increased storage moduli (G′) and loss moduli (G″). Significant increases were noted when PO₄³⁻ concentration was increased, and compared to prior values (*5 mM PO₄³⁻ from Galler et al, G′(--), G″( . . . )). Increasing the density of PO₄³⁻ groups, using the bisphosphonate clodronate (Clod.), resulted in an increase in mechanical strength. Charge density did not result in significantly increased moduli when comparing heparin (Hep.) and trypan blue (Tryp.) at similar concentrations. Charge density and conformation of suramin resulted in an order of magnitude increase in mechanical strength with increase in concentration. Similar Greek letter indicates no statistically significant difference for each receptor (*p<0.01).

FIG. 4 provides data and images related to characterization of peptide structure. (Panel A) FTIR spectroscopy showing characteristic peaks for the formation of a β-sheet structure when peptide is mixed with saline, HBSS, high ionic strength phosphate and two different suramin concentrations. (Panel B) CD spectroscopy confirmed formation of β-sheet structure. Note that gels formed aggregates which resulted in lower minima/maxima intensity for higher concentration suramin. For FT-IR and CD all samples were identically diluted as detailed in the Examples. (Panel C) Peptide-drug mixtures cast in cylindrical molds create optically transparent gels that maintain their structure. (Panel D) Peptide hydrogels with drug loading create nanofiber matrix seen in negative stain TEM, scale bar 50 nm. (E) SEM of dehydrated hydrogel, scale bar 1 μm.

FIG. 5 is a graphical representation of data related to drug release from scaffolds. (Panel A) Short term and long term drug release overlapped. (Panel B) Maximum cumulative suramin concentrations reach 0.6 mg/mL (for high loading) and 0.04 mg/mL (for low loading). (Panel C) Cumulative percentage mass released from scaffolds was determined, modeled, and first derivative release rates determined (Panel D).

FIG. 6 provides a graphical representation of macrophage polarization as a function of suramin loading. Suramin loading hydrogels repressed the expression of M1 macrophage marker CCR7+ (Panel A) and release of pro-inflammatory cytokines (Panel B) TNF-α and (Panel C) IL-1β.

FIG. 7 provides data on in vivo evaluation of one embodiment of the present compositions. (Panel A) Scaffolds with suramin loading show large platelets of MDP uninfiltrated (*), compared to (Panel B) a similar region (*) in SLac only scaffolds. (Panel C) Quantification of infiltration showed a significantly lower number of cells, (Panel D) infiltrating loaded scaffolds to a diminished extent, (Panel E) with a greater M2 macrophage polarization. Representative images of suramin loaded (Panel F) and unloaded (Panel G) scaffolds shown. Nuclei—DAPI: blue, Macrophages: CD68-red, M1: CCR7-green, M2: CD163-purple. (*p<0.01).

### DESCRIPTION

Drug release from polymeric scaffolds for sustained release has been sought after for decades. Several strategies have been attempted for the capture of drugs including dissolving tablets, micelles for hydrophobic drugs, multi-walled microparticles, polymer wafers, covalent immobilization into carriers or onto surfaces, and ionic layer-by-layer self-assembly, to name a few. Of concern is the unfavorable interaction of polymeric carriers with loaded drugs or surrounding tissue, such as: covalent linker addition for conjugation to scaffolds or surfaces which may attenuate the activity or functionality of drugs; non-natural degradation products which may elicit an inflammatory response complicating drug action and causing unwanted side-effects; or the non-conforming nature of solid delivery systems. The present disclosure provides a hydrogel delivery system that addresses these concerns.

The present disclosure is directed to compositions and methods comprising the use of multi-domain peptides (MDPs) that self-assemble into extracellular matrix (ECM) nanofibrous hydrogels to act as a delivery system for therapeutic agents. Generally, MDPs comprise terminally charged residues (first domain) that flank a region of alternating hydrophobic and hydrophilic residues (second domain). These facial amphiphiles associate into anti-parallel β-sheets excluding polar solvents. These self-assembling peptides form short-range fibrils due to molecular frustration between like-like terminal charges. With the addition of therapeutic agents carrying a charge opposed to the terminal charges of the peptides, charges on terminal residues are shielded, allowing long-range fiber growth, entanglement and hydrogel formation. The ionic interaction between the charged therapeutic agent and the MDP hydrogel further aid in sequestration and controlled release of the charged therapeutic agent. Thus, the therapeutic agent not only provides a therapeutic effect on for the subject disease, but also aids in the formation of the hydrogel on which it is carried and released. The interactions that form the drug-loaded hydrogel easily break and reform to allow shear thinning and near instantaneous (within 60 to 90 seconds) recovery (90-95% G′ of initial storage modulus of the peptide structure) thereby permitting aspiration and needle in situ delivery. In sum, the hydrogels formed by the present compositions are injectable, biodegradable, biocompatible, and provide the ability for a controlled release (e.g., extended release) of the associated therapeutic agents.

The peptides of the present compositions comprise a first domain that includes positively or negatively-charged residues that flank the alternating hydrophobic and hydrophilic residues of a second domain. In one embodiment, the terminally charged residues of the first domain include, for example, one to four repeats (i.e. 1-4 amino acids on each end of the second domain) of glutamic acid, aspartic acid, arginine, histidine, or lysine. In one particular example, a single lysine residue is positioned at both the N-terminal and C-terminal ends of the second domain (i.e., the region of alternating hydrophobic and hydrophilic amino acids).

The peptides of the present compositions comprise a second domain that promotes self-assembly of the peptide to a hydrogel structure. In one embodiment, the second domain includes alternating hydrophobic and hydrophilic amino acid residues. Non-limiting examples of suitable hydrophobic amino acid residues includes alanine, leucine, glycine, isoleucine, tryptophan, phenylalanine, proline, methionine, and cysteine. Non-limiting examples of suitable hydrophilic amino acid residues includes serine, tyrosine, threonine, asparagine, and glutamine. The second domain may include two to eight repeats (i.e., 4-16 amino acids) of the hydrophobic/hydrophilic or hydrophilic/hydrophobic sequence. In one embodiment, the second domain comprises six repeats of serine-leucine (12 amino acids).

The peptides of the present composition may optionally an enzymatic cleavage signaling sequence. The enzymatic cleavage signaling sequence of the present disclosure is directed to a sequence that actually signals and results in a specific cleavage event to at least a portion of the peptide content of the composition. For example, the enzymatic cleavage signaling sequence includes sequences specifically recognized by enzymes secreted by tissues or cells, such as macrophages or fibroblasts, invading or surrounding the administration site of the composition. In one embodiment, the enzymatic cleavage signaling sequence is recognized and cleaved by a matrix metalloprotease (MMP) such as those described in Table 1 of Turk, et. al., Nature Biotechnology, 661-667 (2001). In one embodiment, the enzymatic cleavage signaling sequence is susceptible to cleavage by MMP-2 and is leucine-arginine-glycine. The enzymatic cleavage signaling sequence may be separate from the other domains of the peptide or may be embedded in another domain. For example, the cleavage sequence may be placed within the second domain and more specifically, in the middle of the second domain. In the instance the second domain comprises a sequence of six repeats of serine-leucine, the cleavage sequence can be positioned after the third repeat such. For example, a peptide comprising the first domain, second domain, and cleavage sequence may comprises a sequence of KSLSLSLRGSLSLSLK (SEQ ID NO: 2). In this example, the leucine of the cleave sequence -LRG- is provided by the third repeat of the second domain. In other embodiments, the enzymatic cleavage sequence may be between the second domain and fourth domain, within the fourth domain, between the first and second domain (on the C-terminal end), and between the fourth domain and fifth domain.

The peptides of the present compositions further include a spacer. As used herein, the term “spacer” denotes one or more amino acids or a different molecular entity that are generally small and nonpolar in order to minimize the likelihood of interference with self-assembly of the peptide. An amino acid spacer group may include, for example, aminohexanoic acid, polyethyleneglycol, 5 or less repeats of glycine, and 3 or less repeats of glycine-glycine-serine-glycine (SEQ ID NO: 3).

The peptides of the present compositions may further include a cell adhesion sequence. In one embodiment, the cell adhesion sequence comprises -RGD- and may further comprise one or more repeats thereof. Other cell adhesion sequences as known in the art may be substituted such as sequences derived from collagen such as GFOGER (SEQ ID NO: 4), cyclic RGD, other integrin binding sequences.

One example of a peptide to be used in the present compositions, systems, and methods is K-(SL)₂-S(LRG)-(SL)₃-K-G-(RGD)-S(SEQ ID NO: 1) (also referred to herein as “SLac”). This exemplary peptide and others within the scope of the present disclosure assemble into long range nanofibers forming hydrogels.

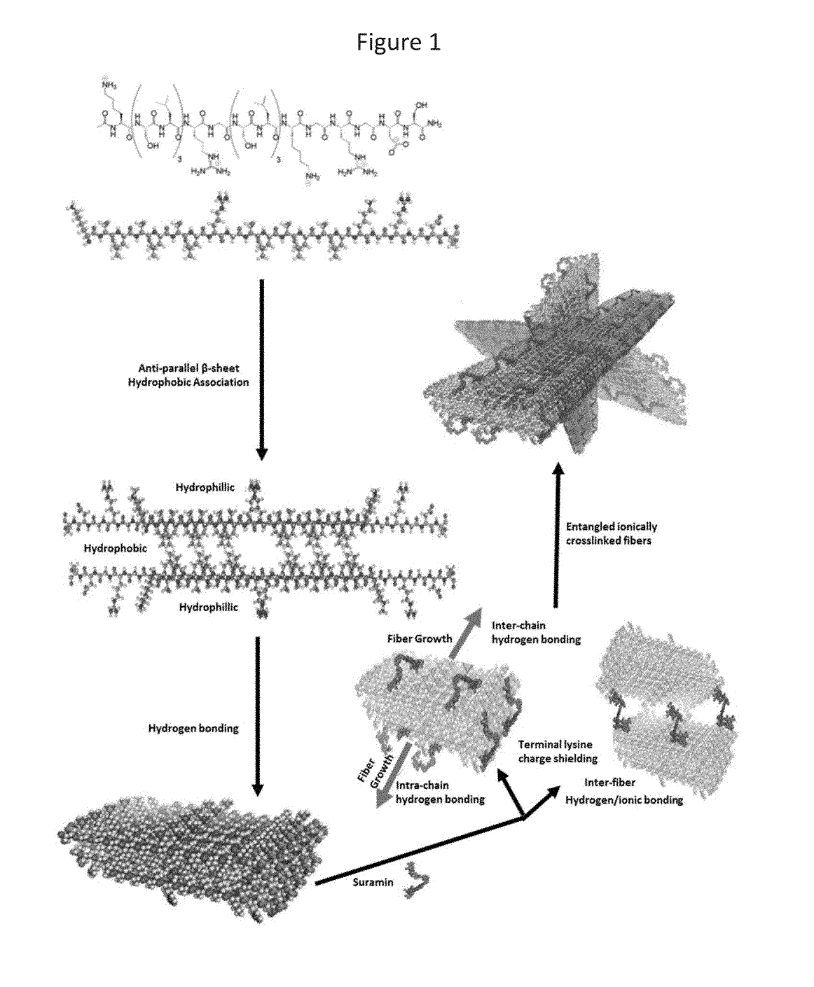

In one embodiment, any of the peptides described above may interact to form a peptide nanofiber. A peptide nanofiber may comprise 4 or more of the above described peptides that interact via hydrophobic interaction between the hydrophobic residues of the second domain and hydrogen bonding of the peptide backbone. FIG. 1 depicts a peptide nanofiber.

The peptides of the present composition can be lyophilized and dissolved in, for example, an appropriate concentration of sucrose solution or in deionized water. In one embodiment, the peptides can be provided in a 1-300 mM sucrose solution. The peptide concentration in the solution may be from about 0.1 mg/ml to about 100 mg/ml, from about 1 mg/ml to about 90 mg/ml, from about 10 mg/ml to about 80 mg/ml, from about 20 mg/ml to about 70 mg/ml, from about 30 mg/ml to about 60 mg/ml, from about 40 mg/ml to about 50 mg/ml, and any concentration therebetween.

The compositions of the present disclosure further comprise a therapeutic agent. The therapeutic agent performs dual functions in the present hydrogel composition. First, therapeutic agent is the active ingredient to treat a particular condition. Thus, it should be understood that the therapeutic agent, as the term is used herein, should not be construed as a docking agent that simply provides a binding site for other active compounds. However, the peptide compositions of the present composition could have multiple other therapeutic agents that are sequestered within the hydrogel or otherwise associated with the hydrogel. Second, the therapeutic agent acts to relieve molecular frustration of the peptide by ionically cross-linking (i.e. electrostatic interaction) with the charged amino acids (first domain) of the peptides described above. This interaction further promotes self-assembly. In the instance the first domain of the peptide amphiphile is a positively charged amino acid, such as lysine, an appropriate therapeutic agent comprises a negative-charge, such as suramin. Conversely, where the first domain is a negatively-charged amino acid, an appropriate therapeutic agent comprises a positive-charge. In the presence of such appropriate charged therapeutic agents, peptides of the present composition self-assembles into β-sheets thereby forming a nanofibrous hydrogel scaffold. Further, given the nature of self-assembly utilizing hydrophobic and hydrophilic interactions and ionic interactions, these bonds easily break and reform on the molecular level thereby demonstrate near instantaneous (less than or about 90 seconds following shear exposure) shear recovery (>90-95% G′) after intermittent high shear events (e.g. 1 minute shear exposure at 100% strain) such as needle aspiration or needle delivery. As such the hydrogel compositions are injectable at high peptide concentrations. This provides a distinct advantage over other hydrogel scaffold systems that do not possess shear recovery and must therefore be implanted as a formed hydrogel and as well from other injectable hydrogel systems that only recover at lower concentrations. As used herein, the term “charged therapeutic agent” is a positively or negatively-charged biological or chemical entity that induces a phenotypic response or molecular or cellular change in an appropriate cell type or tissue when the cell or tissue is contacted with the agent.

In certain embodiments, the therapeutic agent is less than 2,000 Daltons and in other embodiments, is less than 1,000 Daltons. One example of a suitable charged therapeutic agent includes, but is not limited to suramin. Classes of suitable therapeutic agents includes, but is not limited to proteins, glycopeptides, glycosaminoglycans, carbohydrate drugs, lipid based drugs, and small molecules. It should be understood that the compositions and delivery systems of the present disclosure can be used with a variety of different charged therapeutic agents and the present disclosure should only be understood as providing examples of the types of agents that can be employed, and should not be read as limiting on the appended claims directed generally to a “therapeutic agent” or “charged therapeutic agent”.

In one embodiment, the peptides described above provide an extended-release composition. As described above, the peptides of the present disclosure have a domain that comprises charged amino acids (first domain). Oppositely charged drugs of monovalency, divalency, or multivalency can be incorporated into solutions of peptide thereby resulting in ionic interaction between oppositely charged species (peptide and drug) and hence, ionic crosslinking. Ionically cross-linked drug is sequestered by the peptide and matrix to allow for attenuated extended release. This is in contrast to diffusion-based release which is controlled by pore size, tortuosity, material density, pressure, and molecular diffusivity. Conversely, ionically cross-linked drugs have the ability to release as a function of ion exchange and bond breakage. Consequently, ionically cross-linked drug release from polymeric matrices is expected to be delayed compared to diffusion-based release. The release profiles will often depend on the species of polymer and drug released. For example, the release profile of suramin from SLac-based scaffolds is approximately 50% drug release over a 20 day period, and up to 40% retention of drug ionically cross-linked over a period of 30 days.

The present disclosure also provides methods of delivering therapeutic agents to subjects using the peptide compositions described herein. The method comprises administering a therapeutic agent to a target tissue of a subject, wherein the therapeutic agent is administered in anyone of the peptide compositions described herein; and following administration, allowing the composition to form a hydrogel scaffold for delivery of the therapeutic agent ionically cross-linked thereto to the target tissue. For example, the peptide composition of the method may comprise a plurality of peptides ionically cross-linked by the therapeutic agent, wherein each peptide of the plurality of peptides comprises a first and second domain, wherein the first domain is (X)n, where X is a negatively or positively charged amino acid, and n is 1 to 4, wherein the first domain is positioned at both the N-terminal and C-terminal ends of the second domain, and wherein the second domain is (YZ)n′, where Y is a hydrophilic amino acid and Z is a hydrophobic amino acid, or Y is a hydrophobic amino acid and Z is a hydrophilic amino acid, and n′ is 2 to 8, and wherein the therapeutic agent is positively charged where X is negatively charged or wherein the therapeutic agent is negatively charged where X is positively charged. Each peptide may further comprise a spacer, a cell adhesion sequence, and/or an enzymatic cleavage sequence. In certain embodiments, the therapeutic agent is less than 2,000 Daltons, and in other embodiments, the therapeutic agent is less than 1,000 Daltons.

The peptide composition may comprise a peptide nanofiber comprising at least four peptides, wherein each peptide contains a region of hydrophobic amino acids in alternating sequence with hydrophilic amino acids, and a charged amino acid flanking each end of the region, and wherein the therapeutic agent ionically interacts with the charged amino acid. Each peptide may further comprise a spacer, a cell adhesion sequence, and/or an enzymatic cleavage sequence. In certain embodiments, the therapeutic agent is less than 2,000 Daltons, and in other embodiments, the therapeutic agent is less than 1,000 Daltons.

Due to the shear recovery properties of the present compositions, the administering step can be performed through injection with a syringe and needle or some other non-invasive technique that results in sheer thinning of the peptide. Furthermore, the composition can be injected at high final peptide concentrations such as greater than 5 mg/ml to about 50 mg/ml, from about 10 mg/ml to about 20 mg/ml, and all concentrations therebetween. Furthermore, given the injectability, cytocompatibility, biocompatibility, and biodegradability, multiple injections can be performed directly into the target tissue, or proximal and/or distal to, as required, and performed multiple times over periods of seconds, minutes, hours, weeks, months or longer. Additional, the peptide compositions may provide an extended release profile of the associated therapeutic agent such that only a single administration is needed every 14 to 30 days or 21 to 30 days until the subject's condition is resolved or the treatment is otherwise no longer necessary.

In certain embodiments, only a single therapeutic agent is administered via the peptide composition of the present disclosure such that there is not a second therapeutic agent associated with the peptide compositions or with the primary therapeutic agent ionically cross-linked with the peptides. In other embodiments, multiple therapeutic agents may be sequestered or otherwise associated with the peptides for administration.

In certain embodiments, the subject is a human patient suffering from cancer, parasites, or to prevent or treat blot clots or other cardiovascular conditions. Thus, in some embodiments, the target tissue is a solid tumor. In other embodiments, the target tissue may be the site of an injury or wound. In yet another embodiment, the target tissue is the site of active inflammation or other disease activity. The step of administering can be performed by injecting the therapeutic agent cross-linked peptide composition directly on the target tissue or on an adjacent tissue. In certain other embodiments, the step of administering is performed following surgical removal of a solid tumor, and wherein the target tissue is the site from which the solid tumor was surgically removed.

In certain embodiment, prior to the step of administering, the therapeutic agent is combined with the peptide composition and if needed, mixed thoroughly to allow the therapeutic agent to interact with the peptide.

Unless otherwise indicated, all numbers expressing quantities of ingredients, concentrations properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

It is contemplated that any instance, embodiment, or example discussed in this specification can be implemented with respect to any method or composition of the invention, and vice versa. Furthermore, compositions of the invention can be used to achieve the methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

### Examples

To facilitate a better understanding of the present invention, the following examples of specific instances are given. In no way should the following examples be read to limit or define the entire scope of the invention. The following methods and materials were used in the Examples below.

Peptide Design and Characterization:

Multidomain peptides were designed with the following sequence (SLac): K-(SL)₃-(RG)-(SL)₃-K-GRGDS (SEQ ID NO: 1). Standard solid phase peptide synthesis was performed on Apex Focus XC using Rink amide resin with 0.37 mM loading and N-terminal acetylation. Post cleavage from resin, crude mass was checked prior to dialysis with 500-1200 MWCO dialysis tubing against MilliQ water. Peptides were subsequently lyophilized, confirmed for purity using time-of-flight electrospray ionization mass spectrometry, MicroTOF ESI, and reconstituted at 20 mg/mL in sterile 298 mM sucrose.

Peptide (Nanofiber)-Drug Interaction:

Peptides were modeled in PyMOL Molecular Graphics System, Version 1.5.0.5 based on previous published work. As shown in FIG. 1, the peptide chains form an anti-parallel β-sheet that sequesters the non-polar residues away from water. Hydrogen-bonding between the sandwiched β-sheets facilitates one-dimensional propagation in nanofibers. The anti-parallel β-sheet arrangement of peptides in SLac was confirmed using Fourier-Transform Infrared Spectroscopy (FTIR). For modeling in PyMOL, 8-10 peptide chains were arranged adjacently in an anti-parallel fashion with hydrophobic/hydrophilic faces (dependent on amino acid R-chain) facing the same direction. The two hydrophobic faces were brought in proximity of each other as suggested by previous studies. Ionic interactions between facial amphiphilic fibers were then hypothesized with suramin.

Scanning Electron Microscopy (SEM) and Transmission Electron Microscopy (TEM):

Microscopic morphology of SLac scaffolds gelled with suramin was determined using SEM and TEM. For SEM, samples were ethanol dehydrated, critical point dried, sputter coated with 7 nm gold, and imaged using a FEI Quanta 400 ESEM. Fibrillar network for SLac was confirmed using TEM. For TEM, samples were prepared by adding peptide solution directly onto Quantifoil R1.2/1.3 holey carbon mesh on copper grids. Excess peptide was blotted and the grid was dried prior to negative staining using 2.0% pH 7 phototungstic acid for 8 min. Dried grids were imaged using a JEOL 2010 microscope (120 kV).

Circular Dichroism:

All circular dichroism experiments were performed on a Jasco J-815 spectropolarimeter equipped with a Peltier temperature controlled stage. All spectra were collected from 190-250 nm at 25° C. Peptide was dissolved in sucrose at 2 wt %. CD samples were prepared at pH 7.4 by gelling peptide in equal volumes of 0.9% saline, HBSS, 7.7 mM PO₄³⁻, 1.0 mg/mL suramin or 0.1 mg/mL suramin. For each experiment, 20 μL aliquots of each sample were gelled in situ on a Quartz cell with a path length of 0.01 cm. The molar residual elipticity (MRE) was calculated from the following formula:

[θ]=(θ*m)/(C*nᵣ*l)

where θ is the observed ellipticity in millidegrees, m is the molecular weight in g-mol⁻¹, C is the concentration in mg-ml⁻¹, l is the path length of the cuvette in cm, and nᵣ is the number of amino acids in the peptide.

Attenuated Total Reflectance Infrared Spectroscopy (ATR-IR):

10 μL aliquots of peptide gel at pH 7.4 was pipetted onto a “Golden Gate” diamond window and dried under nitrogen until a thin film of peptide was achieved. IR spectra (64 accumulations) were taken using a Jasco FT/IR-660 spectrometer.

Mechanical Analysis:

Peptide solutions were made by dissolving lyophilized SLac in 298 mM sucrose-water at a concentration of 2 w %, pH 7.4. Hydrogels were constructed by addition of Hank's balanced salt solution (HBSS) at a 1:1 ratio. Multivalent drugs were added at similar concentrations, approximating physiological doses, to SLac solutions with final concentrations of: SLac—0.4 mM (1 w %); concentration of positive lysine charges—0.8 mM; suramin—3.8 mM (0.5 w %) or 0.38 mM (0.05 w %); trypan blue—3.2 mM (0.2 w %); heparin ˜2.78 mM (5 w %); clodronate disodium—3.4 mM; phosphate buffer—38 mM and 5 mM; and 0.9% saline. Negatively charged polyvalent ions in buffer solution facilitated intermolecular ionic interactions with lysine residues, crosslinking the hydrogel. Rheological behavior of peptide hydrogels was determined using an 8 mm parallel plate geometry at a gap of 250 μm. 50 μL of hydrogel was constructed and, within 30 min, placed on stainless steel plates of a rheometer. A frequency sweep (0.1-100 Hz, at constant 1% strain), strain sweep (0-1000%/strain, at 1 Hz) and shear recovery (1% strain for 30 min, 100% strain for 60 s, 1% strain for 30 min) were performed. We ensured the phase angle δ≦90° to ensure no slipping.

Suramin Drug Loading and Release:

Suramin was dissolved in 0.9% saline and loaded into MDP hydrogel (100 μL of SLac dissolved in 298 mM sucrose+100 μL of drug/saline) in microcentrifuge tubes. 1 mL of release media (HBSS) was added to gels placed in a humidified 5% CO2 cell culture incubator at 37° C. 200 μL aliquots of release media were assayed, with replenishment, daily for 30 days. To determine short term release kinetics, in a separate setup, aliquots were assayed at 1, 2, 4, 8, 12, 24, 48 and 72 hr. Concentration of drug was determined using UV spectrophotometry at 313 nm, n=5. Mass release data were plotted as a function of cumulative release of drug in Sigmaplot. Drug release was modeled using the Korsmeyer-Peppas equation.

Monocyte/Macrophage Culture and Differentiation:

Human monocytic leukemia cell line, THP-1 cells, were cultured in media (ATCC-formulated RPMI-1640 Medium) supplemented with 0.05 mM 2-mercaptoethanol, fetal bovine serum (10%), 100 mg/ml penicillin, and 100 mg/ml streptomycin at a concentration of 200,000 cells/mL. Cells were grown in suspension and diluted when concentration reached 0.8-1.0 million cells/mL. Media was changed every 3 days, as necessary. THP-1 mono-cytes were cultured to M₀ macrophages by pulsing with 5 nm phorbol 12-myristate 13-acetate, PMA for 5 mins. Adherent M₀ differentiated cells were incubated with IFN-γ (20 ng/mL+LPS (20 ng/mL) for M₁ for 24 hours at 37° C. For macrophage plasticity studies, THP-1 cells were differentiated to M₀ macrophages as described. 0.5M cells/cm2 were seeded atop 100 μL of SLac hydrogels made with 1 w % suramin (Final: 1 w % Peptide, 0.5 w % suramin), 0.1 w % suramin (Final: 1 w % Peptide, 0.05 w % suramin) or PBS (Final: 1 w % peptide), n=5, in 16 well glass bottom glass slides. Media containing IFN-γ (20 ng/mL)+LPS (20 ng/mL) was added atop hydrogels to stimulate M₁ phenotype. Control hydrogels were gelled using PBS. M₀ control did not receive IFN-γ/LPS, positive control media was supplemented with 0.5 mg or 0.05 mg of suramin (identical to mass of suramin in gels). After 24 hr, media aliquots were stored at −80° C., and proinflammatory markers IL-1β and TNF-α were measured. Gels were then fixed in formalin and immunostained for M1 marker CCR7 and DAPI. Number of CCR7+ cells divided by number of nuclei gave M₁ polarization ratio quantified by counting 6 random image sections at 20× magnification per sample, of 5 samples per group, using NIH Image J.

Subcutaneous Implants:

All experiments were approved by the Rice University Institutional Animal Care and Use committee. Female Wistar rats (225-250 g) were anesthetized using isofluorane (2% for induction and 1% for maintenance), dorsal aspect shaved and sterilely prepped. Scaffolds were loaded in syringes and 200 μL subcutaneous injections of each SLac and SLac+suramin (100 mg/mL) were made in 4 different 1.5 inch spaced randomized sites on the dorsal aspect, on either side between the lower thoracic and upper lumber vertebrae, n=4 for each scaffolds for 1 week. Rats were then euthanized using an overdose of isoflurane, CO₂ asphyxiation, and bilateral thoracic puncture. The dorsal skin was removed around the entire implant, washed with PBS, and fixed in neutral buffered formalin for 24 hr prior to processing. Tissue was then processed into paraffin blocks, sectioned at 7 μm, deparraffinized and stained for cellular infiltrate using hematoxylin and eosin (H&E), and nuclei in 3 random fields per sample and averaged over all samples from each group were counted using Image J. Infiltration of implants was graded on a 5 point scale: 1—periphery (<50%, with large parts of scaffold uninfiltrated, center uninfiltrated); 2—50-80% (with small regions of scaffold exposed, center uninfiltrated); 3—center infiltrated (with small regions of scaffold exposed); 4—few to no scaffold regions visible; 5—implant indistinguishable from native tissue except for complete dense cellular repopulation. Cellular infiltrate was phenotyped using immunostaining. Immune cell staining was performed for 1) pan-macrophage rabbit anti-rat CD68, M₁ macrophages goat anti-rat CCR7, M₂ macrophages mouse anti-rat CD163. Secondary antibodies used were: 1) AF647 donkey anti-rabbit, 2) AF488 donkey anti-goat, 3) AF555-donkey anti-mouse. Nuclei were counterstained with DAPI. Cellular infiltrate was quantified using NIH Image J and M₁/M₂ polarization ratio was determined.

### Example 1: Rheological Properties of Crosslinked Peptide Hydrogels

To determine material drug interactions, we first noted the propensity for MDP to form robust hydrogels when like-charges were shielded by negative ions, such as PO43-, allowing fiber crosslinking (FIG. 1).

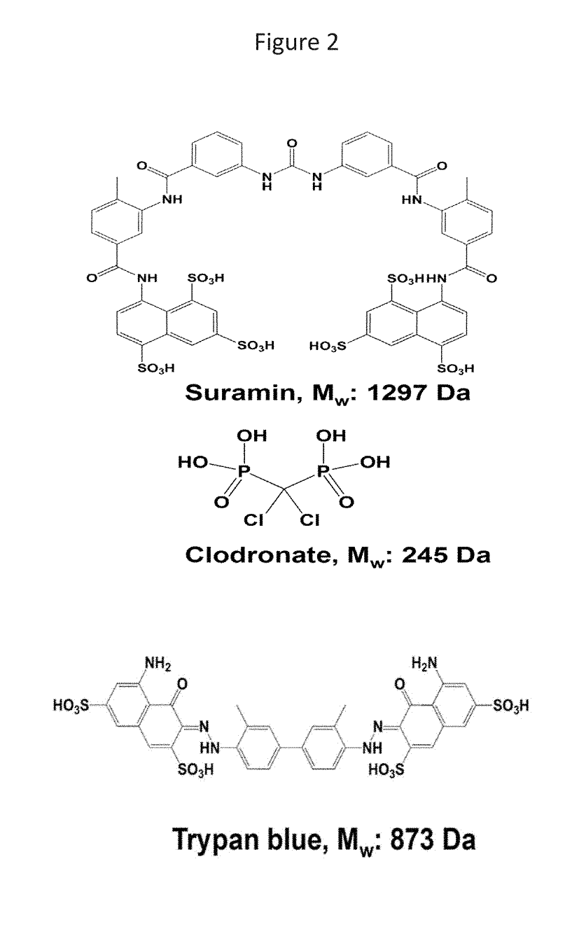

Extending from this, we noted the potential for a variety of anionic drugs to participate in a similar role, while potentially providing higher mechanical strength due to their con-formation, molecular weight and charge density (FIG. 2). To determine which of these strategies would best crosslink MDP, we used parallel plate rheometry.

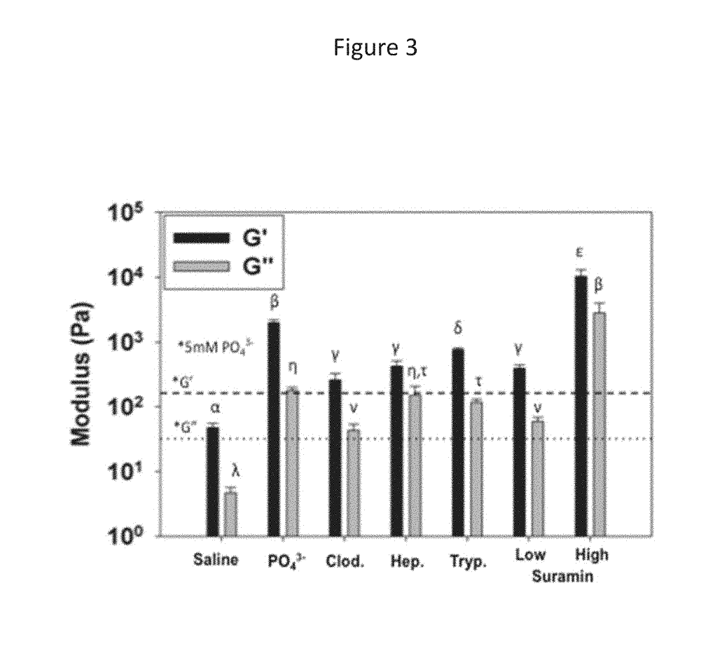

Rheological characterization of hydrogel scaffolds when crosslinked with multivalent drugs may help elucidate potential mechanisms for crosslinking. Similar therapeutic doses of drugs were chosen for loading into hydrogels. Therapeutic doses approximate the concentration of ‘low suramin’. ‘Low suramin’ to ‘high suramin’ loading spanned the range of concentrations that would diffuse to tissue or be bolus dosed. All multivalent drugs showed a marked increase in G′ and G″ as a function of concentration (p<0.01) (FIG. 3). Given the high G′ and G″, the gels created were easily handleable and manipulable. Uniquely, depending on the crosslinking molecule, distinct rheological properties were noted. Increasing PO₄³⁻ buffer ionic strength from 5 mM to 38.5 mM resulted in a significant increase in mechanical strength. Extending this notion of phosphate based crosslinking, a clinically relevant diphosphonate used for bone healing—clodronate disodium, also resulted in a distinct increase in G′/G″ over phosphate alone or saline. The two phosphonate groups of clodronate are capable of shielding a greater number of charges and creating crosslinks to multiple MDP. To test this hypothesis, heparin, a large molecule with several negative charges, was chosen next. Heparin loaded gels showed a significant increase in mechanical strength over PO₄³⁻ but not clodronate alone. Further investigating functionality, a poly-sulfonate—trypan blue, typically used in cell culture viability assays, crosslinked MDP with a similar distinct increase in G′/G″. However, increases in mechanical responses yield gels of similar strength to those crosslinked with heparin. Finally, suramin with 6 sulfonate groups, at a similar concentration, showed a much higher increase in strength (an order of magnitude higher G′/G″) as compared to other drugs used. While the concentration and total number of charges in heparin gels is the greatest, gels prepared with suramin displayed the highest increase in strength (highest G′ and G″) (FIG. 3). This suggests that suramin has a conformationally and structurally suitable architecture in the presence of MDP to aid in its shielding of charges and crosslinking.

### Example 2: Characterization of Peptide Structure

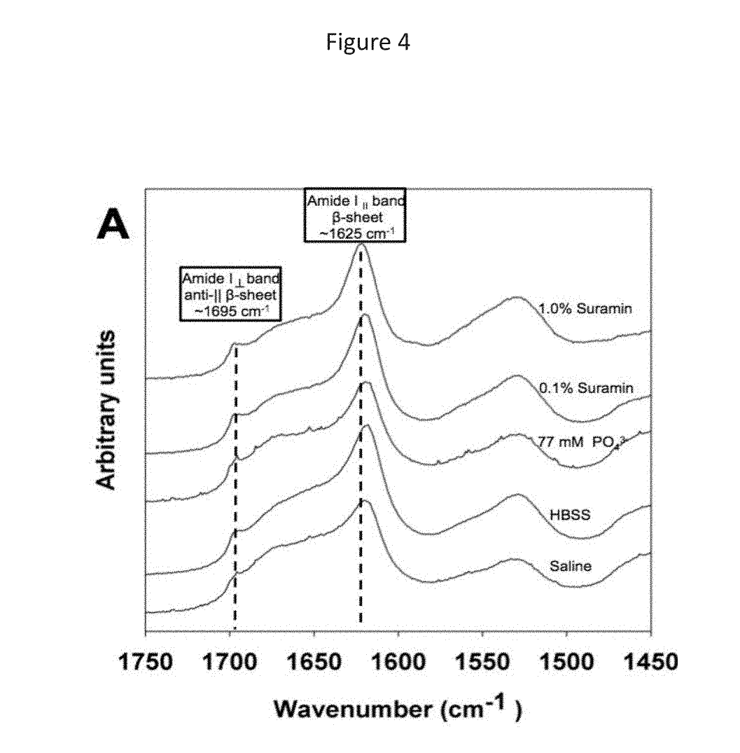

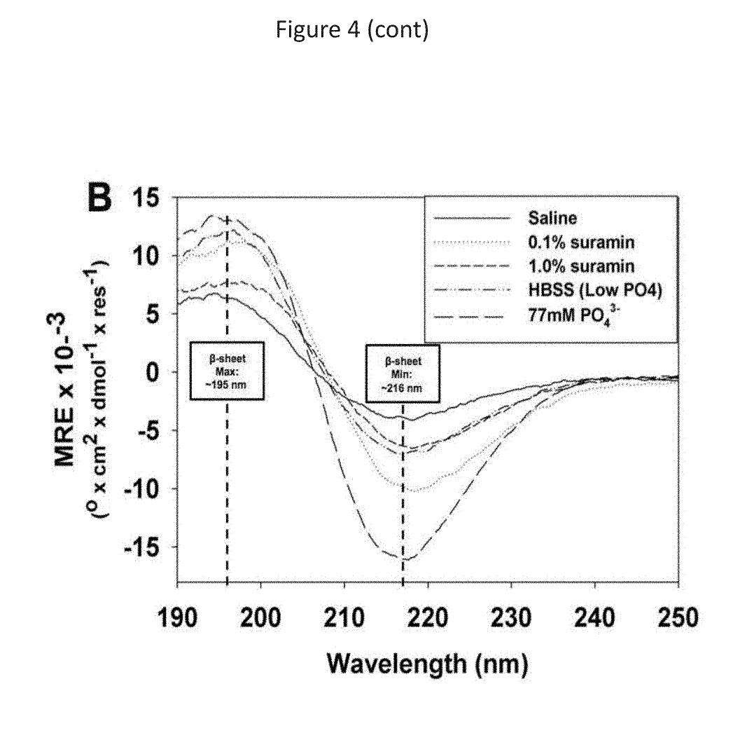

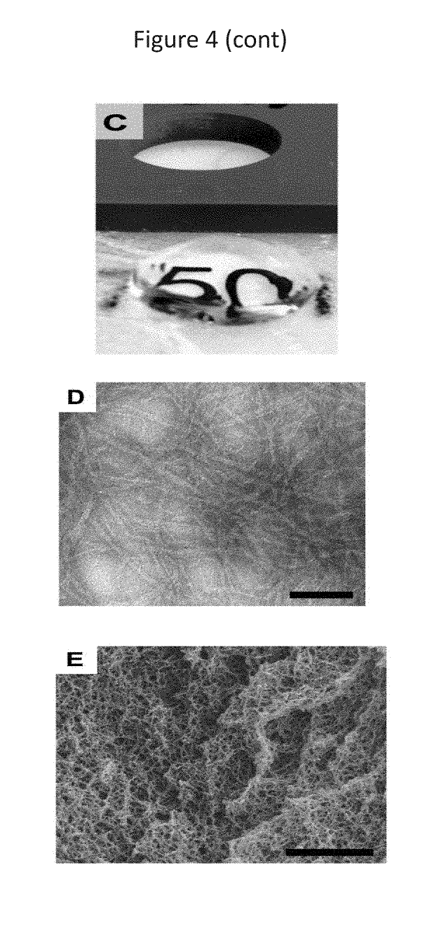

Basic modeling of the multivalent drug-SLac interaction suggest that at neutral pH deprotonated charged groups can interact with several positively charged terminal lysine amine groups, reducing charge-charge repulsion, and promoting intra- and inter-peptide crosslinking. Extended to suramin, we hypothesize that negatively charged sulfonate residues crosslink positively charged lysine side chains (FIG. 1). FT-IR spectroscopy of hydrogels showed characteristic extended β-sheet hydrogen bonding peak between 1610-1630 cm-1, and characteristic antiparallel β-sheet 1695 cm-1 peak for all peptide mixtures (FIG. 4, panel A). CD spectra similarly showed the presence of β-sheet secondary structure, with a minimum around 216 nm and maximum around 195 nm (FIG. 4, panel B). Formation of fibrous structure within hydrogels at high concentrations of suramin/high ionic strength, resulted in lower peak magnitudes at the 195 nm maxima. Peptide hydrogels that formed were optically clear and conformed to the shape of the mold they were cast in (FIG. 4, panel C). Microstructure of peptide scaffolds, probed using TEM and SEM, showed formation of a nanofibrous matrix (FIG. 4, panels D-E).

### Example 3: Controlled Release of Ionically Sequestered Drug

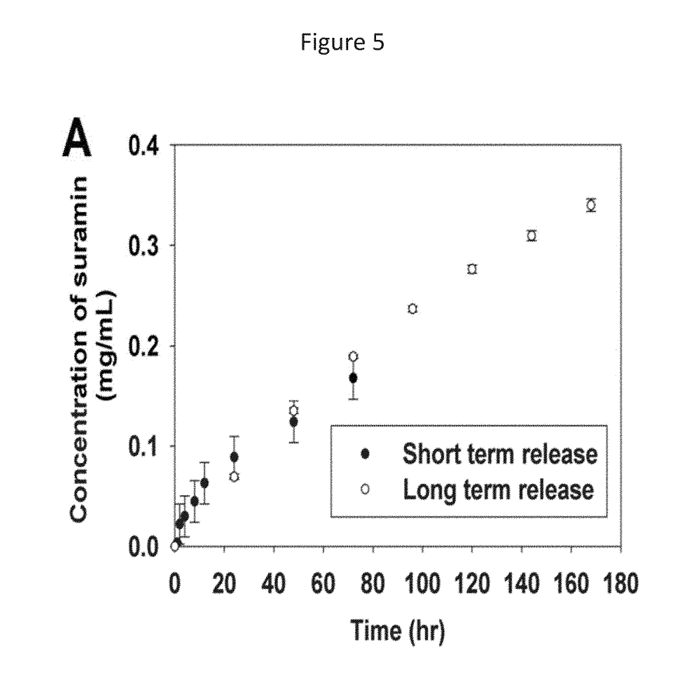

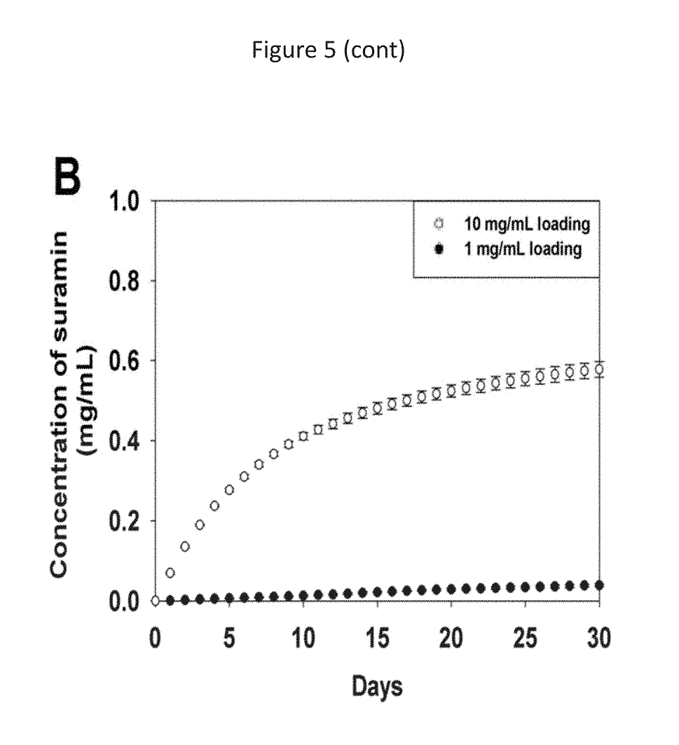

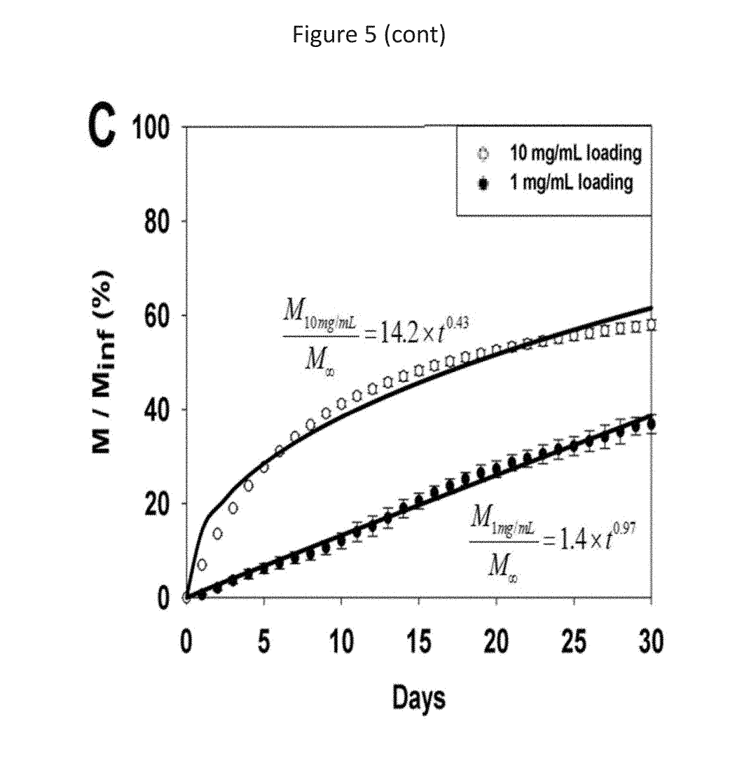

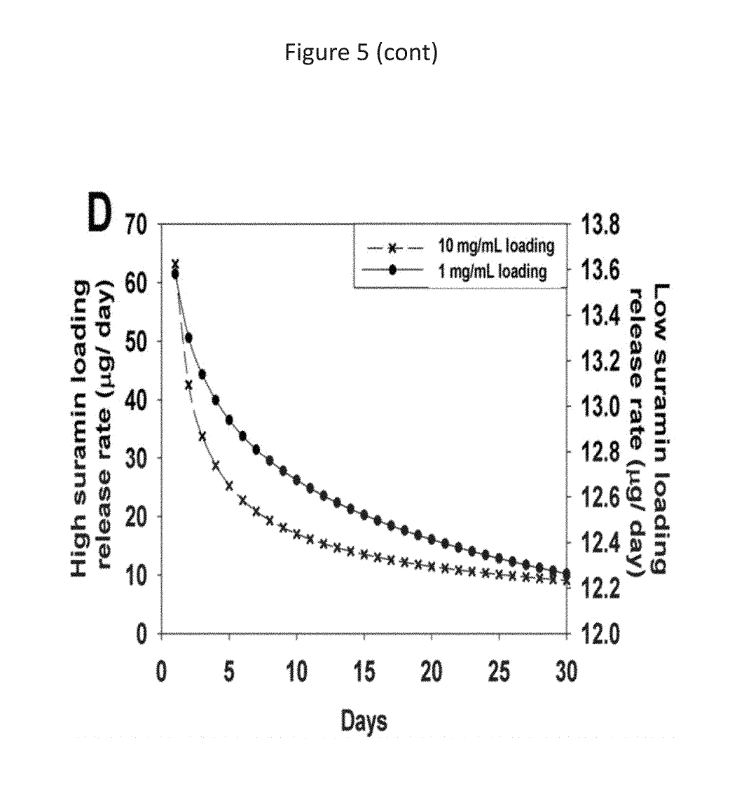

Previous studies have shown the ability to tailor MDP with unique functionality based on peptide sequence. Additionally, MDP can be loaded with drugs, growth factors, cytokines and cellular secretome. As noted, chemical crosslinking of drugs with subsequent controlled release can strongly promote localized tissue responses, and obviate systemic side effects. To this end, the potential of suramin as a potent anti-angiogenic, anti-neoplastic and anti-microbial drug to serve as a chemical crosslinker as demonstrated above was noted. Given suramin's high IV dosing, and frequency of dosing (1 g every/3-7 days in adults), delivery of suramin in situ may prove to be advantageous. However, concerns exist over retention of suramin at the delivery site and leakage of the drug into the lymphatic circulation. This may be reduced by ionically crosslinked suramin sequestered within hydrogels with slow, steady release (FIG. 1, FIG. 5). Suramin crosslinked gels showed long-term release at both low and high concentrations, with much of the drug still present in the carrier hydrogel at 30 days (FIG. 5). At high suramin loading (1.0 mg suramin, 2.0 mg peptide) hydrogel scaffolds showed an initially linear cumulative mass release followed by a tapering off to 57.9±1.1% with the rest of the drug (42.1%) remaining trapped within the hydrogel. At the lower suramin loading (0.1 mg suramin, 2.0 mg peptide), hydrogel scaffolds exhibited significantly lower cumulative release, 38.7±3.2%, with the rest of the drug (61.3%) remaining trapped within the hydrogel (FIG. 5, panels B-C). Modeling of suramin release from polymers was performed to help determine the mechanism of release.

Release from 10 mg/mL loaded gels was diffusion dependent, t≈0.5, R²=0.972. Release from 1 mg/mL loaded gels was non-Fickian diffusion/Case II transport dependent, t≈1.0, R²=0.993, suggesting erosion of hydrogel ionic crosslinks was releasing suramin. First derivatives of release profiles showed that release rates were initially faster for both loading concentrations, which then tapered after 2 weeks to a near linear release rate (FIG. 5, panel D). The aggressive design of the release experiment, with release being probed daily, may overestimate release from hydrogel scaffolds, as diffusive and convective transport enhances drug release and the suramin reservoir concentration in the release media is depleted, however we assayed release aliquots over a shorter time period more frequently (hr vs. day) and determined no significant difference in kinetics of release (FIG. 5, panel A). Given the release data, we hypothesize that the immediate micro-environment will be affected by low concentrations of the drug over the first 30 days, with much of the suramin release being dictated by cellular adhesion to MDP hydrogels, mediated by -RGDS terminal sequence (SEQ ID NO: 5), and degradation of MDP matrix, mediated by MMP susceptible -LRG- domain. Sustained and targeted delivery of suramin has been a goal of several groups; liposomal encapsulation of suramin for antiviral applications, encapsulation of suramin/paclitaxel into PLLA/PLGA microparticles for cancer treatment, and local delivery to inhibit neointimal hyperplasia post-angioplasty to name a few. However, of primary concern with any of these techniques is the release, detailed above, and bioavailability, detailed below, of the suramin after loading into the carrier.

### Example 4: Preservation of Biological Activity in Ionically Cross-Linked Gels

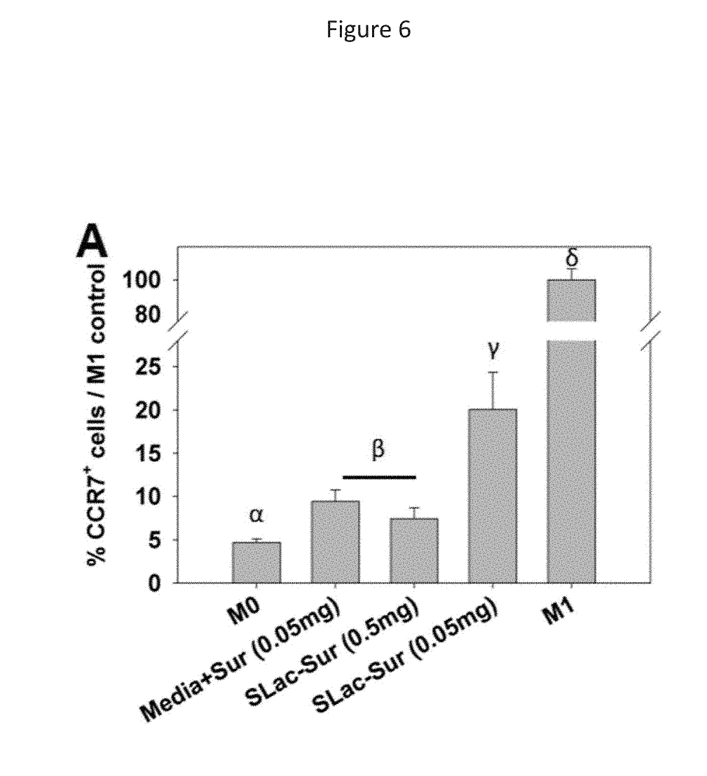

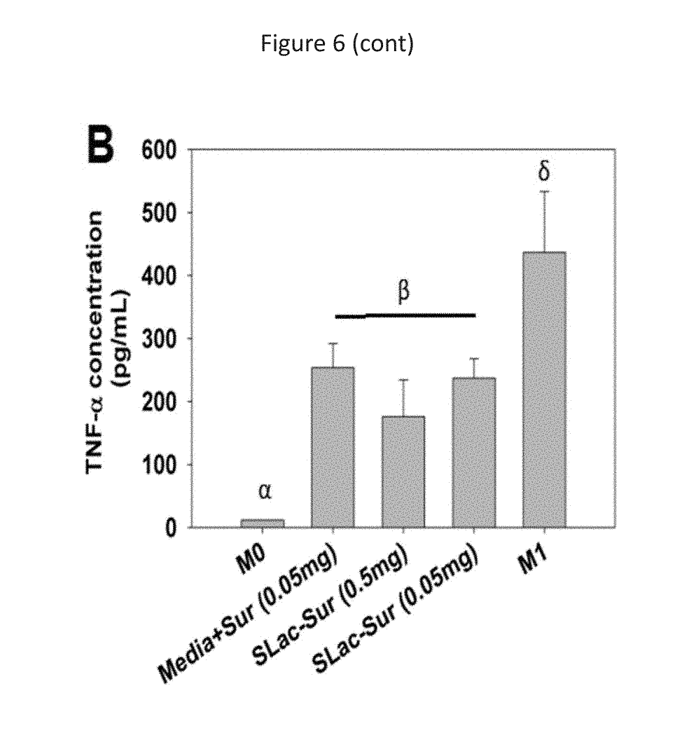

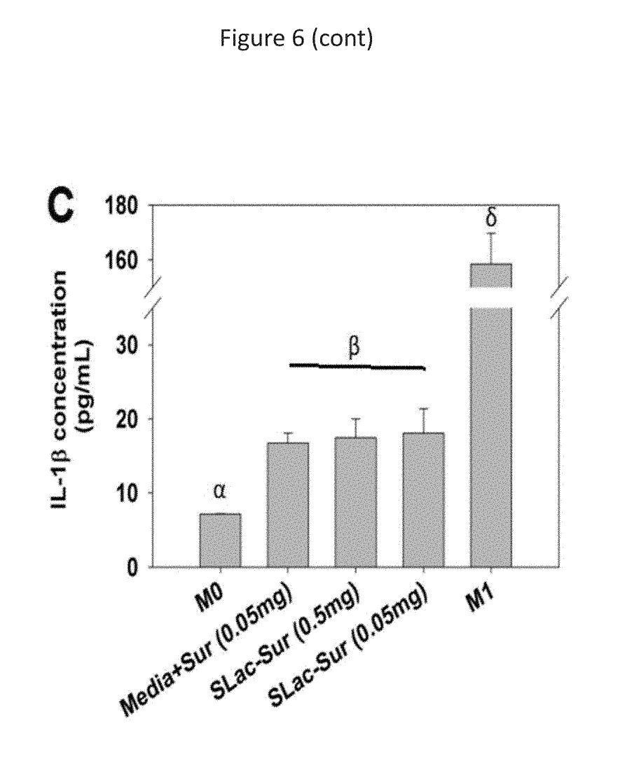

Several strategies for drug release from polymeric scaffolds for sustain release have been attempted for the capture of drugs including dissolving tablets, micelles for hydrophobic drugs, multi-walled microparticles, polymer wafers, covalent immobilization into carriers or onto surfaces, and ionic layer-by-layer self-assembly, to name a few. Of concern is the unfavorable interaction of polymeric carriers with loaded drugs or surrounding tissue, such as covalent linker addition for conjugation to scaffolds or surfaces which may attenuate activity of functionality of drugs; or non-natural degradation products which may elicit an inflammatory response complicating drug action, or the non-conforming nature of solid delivery systems. In this Example we have explored the potential for a drug to actively interact with its carrier—crosslinking it. As a vital next step, it was important to confirm the maintenance of activity of ionically sequestered drugs after loading into MDP carriers. Since suramin is known to have distinct effects on growth factors/growth factor receptors, we utilized suramin releasing gels to determine biological activity. Suramin loaded SLac hydrogels were seeded with THP-1 macrophages. THP-1 cells were chosen given their neoplasticity, monocytic origin and as an established human cell line. LPS activated THP-1 macrophages adhered to drug laden SLac hydrogels. These cells were exposed to ionically trapped suramin, which decreased M₁ polarization with decreased levels of TNF-α and IL-1β compared to M₁ cells (FIG. 6). This is probably due to known P2Y receptor antagonism, as reported previously. Together this data demonstrates preservation of suramin effector functionality, and inhibition of pro-inflammatory M₁ phenotype development in LPS induced human monocytic leukemia cell line THP-1 cells.

### Example 5: Measuring In Vivo Activity

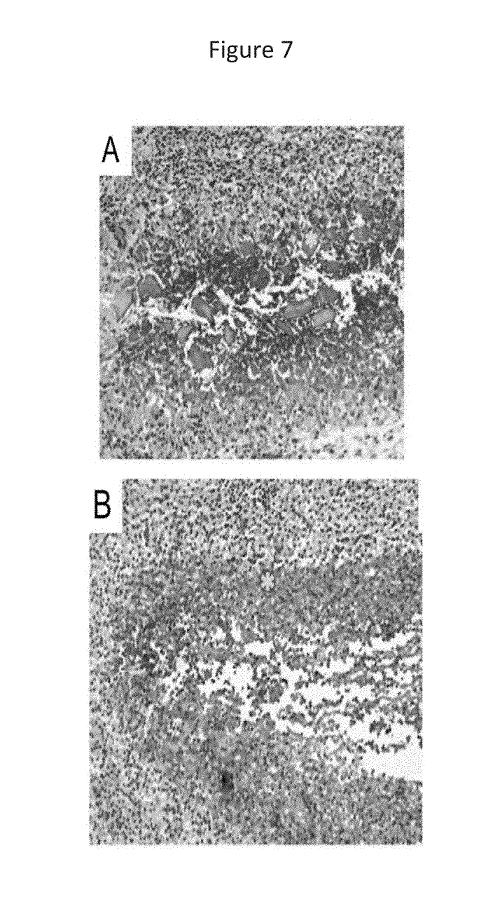

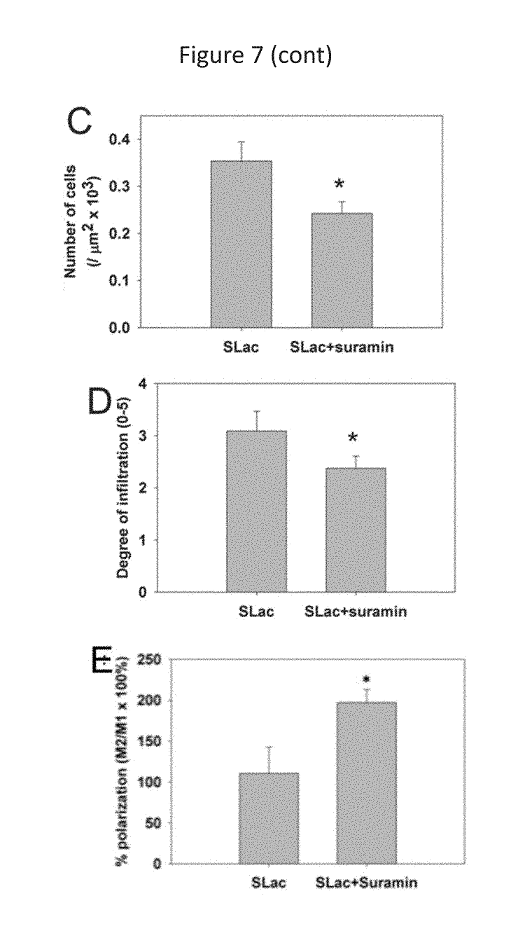

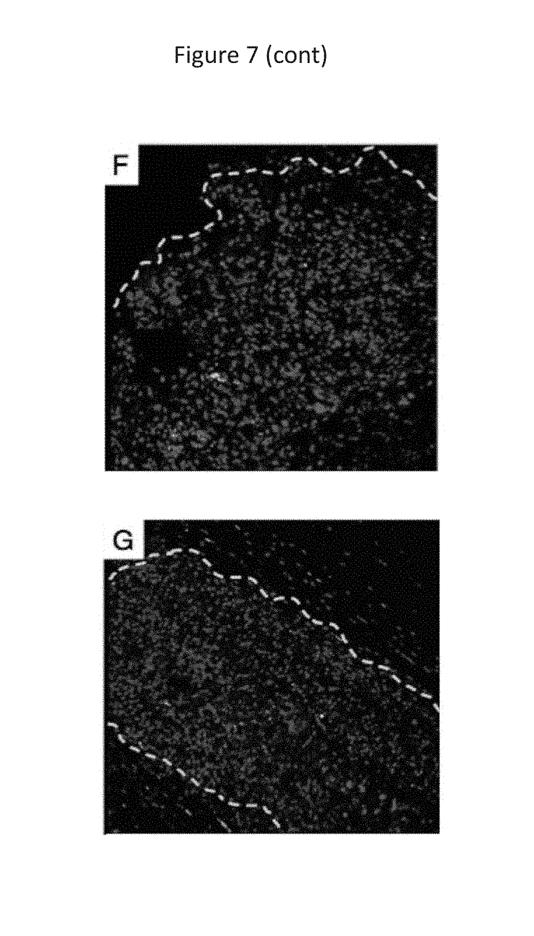

Suramin-crosslinked SLac hydrogels may have potential for localized drug delivery. To demonstrate this, high concentration suramin was used to crosslink 1 w % SLac hydrogels. Suramin crosslinked, compared to PO₄³⁻ crosslinked, gels showed significantly higher stiffness, G′/G″ (FIG. 3). However, hydrogels were still injectable, allowing site delivered subcutaneous implantation. In FIG. 7, suramin hydrogels showed a marked decrease in cellular infiltration compared to PO₄³⁻ crosslinked SLac hydrogels. This may potentially be due to the increased stiffness of matrices that encumber cellular infiltration. Further a significant increase in immunostained M₂ macrophages was observed in suramin gels compared to unloaded gels. MDP hydrogels have previously demonstrated the lack of a fibrous capsule and excellent ECM integration. Suramin loaded hydrogels show a similar lack of fibrous encapsulation and excellent integration as demonstrated in FIG. 7. These results demonstrate the minimal effect implanted gels have on surrounding tissue. Specifically: i) cellular infiltrate was localized to the implant, ii) M₂ macrophage polarization was localized to within the implant and the immediate vicinity, iii) no fibrous tissue walling off the implant was observed. These syringe deliverable constructs may help a variety of strategies that allow localized drug delivery, complemented by functional peptide signaling, that ultimately offer another tool for targeted therapeutics.

In these Examples, we have demonstrated the ability of charged drugs to ionically crosslink multidomain peptides. Specifically, we modeled interactions of peptides with drugs, showing stabilization of an anti-parallel β-sheet structure which enhanced long-range nanofibrous meshwork formation, and mechanically robust gels. We further demonstrated cross-linking of scaffolds using a variety of clinically relevant charged drugs such as suramin, clodronate, heparin and trypan blue. Sequestered suramin was shown to slowly release from hydrogel scaffolds, with less than 40-60% releasing over the first 30 days, depending on loading concentration. Preservation of suramin activity on attenuation of M₁ phenotype of LPS stimulated THP-1 monocytic leukemia cells was demonstrated.

The present invention is well adapted to attain the ends and advantages mentioned as well as those that are inherent therein. It should be understood, however, that the description of specific example embodiments is not intended to limit the invention to the particular forms disclosed, but on the contrary, this disclosure is to cover all modifications and equivalents as defined by the appended claims. While numerous changes may be made by those skilled in the art, such changes are encompassed within the spirit of this invention as illustrated, in part, by the appended claims.

What is claimed is:

1. A composition comprising:
a plurality of peptides ionically cross-linked by a therapeutic agent, wherein each peptide of the plurality of peptides comprises a first and second domain, wherein the first domain is (X)ₙ, where X is a negatively or positively charged amino acid, and n is 1 to 4, wherein the first domain is positioned at both the N-terminal and C-terminal ends of the second domain, and wherein the second domain is (YZ)_{n′}, where Y is a hydrophilic amino acid and Z is a hydrophobic amino acid, or Y is a hydrophobic amino acid and Z is a hydrophilic amino acid, and n′ is 2 to 8; and
wherein the therapeutic agent is positively charged where X is negatively charged or wherein the therapeutic agent is negatively charged where X is positively charged, wherein the therapeutic agent ionically interacts with X, and wherein the therapeutic agent is less than 2000 daltons.

2. The composition of claim 1 wherein the therapeutic agent is less than 1000 daltons.

3. The composition of claim 1 wherein the therapeutic agent is suramin.

4. The composition of claim 1 wherein there is not a second therapeutic agent associated with the plurality of peptides or with the therapeutic agent.

5. The composition of claim 1 wherein each peptide further comprises a cell adhesion sequence.

6. The composition of claim 6 wherein the cell adhesion sequence is RGD.

7. The composition of claim 1 wherein each peptide further comprises an enzymatic cleavage signaling sequence.

8. The composition of claim 7 wherein the enzymatic cleavage signaling sequence is leucine-arginine-glycine.

9. The composition of claim 1 wherein each peptide further comprises a spacer.

10. The composition of claim 9 wherein the spacer is selected from the group consisting of aminohexanoic acid, polyethyleneglycol, 5 or fewer glycine residues, and 3 or fewer of the sequence glycine-glycine-serine-glycine (SEQ ID NO: 3).

11. The composition of claim 1 wherein each peptide further comprises a cell adhesion sequence, an enzymatic cleavage signaling sequence, and a spacer.

12. The composition of claim 1 wherein X is selected from the group consisting of glutamic acid, aspartic acid, arginine, histidine, and lysine.

13. The composition of claim 1 where the sequence of each peptide is SEQ ID NO: 1.

14. A composition comprising a peptide nanofiber comprising at least four peptides, wherein each peptide contains a region of hydrophobic amino acids in alternating sequence with hydrophilic amino acids, and a charged amino acid flanking each end of the region; and a charged therapeutic agent ionically interacting with the charged amino acid, wherein the charged therapeutic agent is less than 2000 daltons.

15. A method for delivering one or more therapeutic agents to a subject comprising administering an extended-release composition comprising a therapeutic agent to a target tissue of the subject, wherein the therapeutic agent is less than 2000 daltons, wherein the extended-release composition comprises a plurality of peptides ionically cross-linked by the therapeutic agent, wherein each peptide of the plurality of peptides comprises a first and second domain, wherein the first domain is (X)ₙ, where X is a negatively or positively charged amino acid, and n is 1 to 4, wherein the first domain is positioned at both the N-terminal and C-terminal ends of the second domain, and wherein the second domain is (YZ)_{n′}, where Y is a hydrophilic amino acid and Z is a hydrophobic amino acid, or Y is a hydrophobic amino acid and Z is a hydrophilic amino acid, and n′ is 2 to 8, and wherein the therapeutic agent is positively charged where X is negatively charged or wherein the therapeutic agent is negatively charged where X is positively charged, and wherein the therapeutic agent ionically interacts with X.

16. The method of claim 15 wherein each peptide further comprises a cell adhesion sequence, an enzymatic cleavage signaling sequence, and a spacer.

17. The method of claim 16 wherein the enzymatic cleavage signaling sequence is leucine-arginine-glycine.

18. The method of claim 16 wherein the spacer is selected from the group consisting of aminohexanoic acid, polyethyleneglycol, 5 or fewer glycine residues, and 3 or fewer of the sequence glycine-glycine-serine-glycine (SEQ ID NO: 3).

19. The method of claim 16 wherein the cell adhesion sequence is RGD.

20. The method of claim 1 where the sequence of each peptide is SEQ ID NO: 1.
